(19) United States
(12) Patent Application Publication
(10) Pub. No.: US 2015/0165421 A1
(43) Pub. Date: Jun. 18, 2015

(54) MOLECULAR SIEVE CATALYST AND PROCESSES FOR FORMULATING AND USING THE SAME

(71) Applicant: SHELL OIL COMPANY, Houston, TX (US)

(72) Inventors: Marianne Hendrina Francisca Kox, New Orleans, LA (US); Jeroen Van Westernen, Amsterdam (NL)

(21) Appl. No.: 14/368,532

(22) PCT Filed: Dec. 20, 2012
(86) PCT No.: PCT/EP2012/076432

§ 371 Date: Jun. 25, 2014

(30) Foreign Application Priority Data
Dec. 27, 2011 (EP) 11195816.1

(51) Int. Cl. B01J 29/06; C07C 1/20; B01J 21/08; B01J 29/70; B01J 29/40

(52) U.S. Cl. CPC B01J 29/06; B01J 29/7046; B01J 29/7042; B01J 29/40; B01J 21/08; C07C 1/20; C07C 2529/06

(57) ABSTRACT

The present invention provides a molecular sieve catalyst which comprises a zeolitic material, a binder material and a matrix material, wherein the zeolitic material comprises zeolite and crystobolite, and the crystobolite is present in an amount of less than.0 wt %, based on the total weight of zeolitic material. The present invention further provides a process for formulating the molecular sieve catalyst, and processes for the preparation of olefins.

### FIELD OF THE INVENTION

This invention relates to a molecular sieve catalyst and processes for the preparation of olefins such as ethylene and propylene.

### BACKGROUND OF THE INVENTION

Processes for the preparation of olefins are known in the art.

U.S. Pat. No. 6,797,851 describes a process for making ethylene and propylene from an oxygenate feed using two or more zeolite catalysts.

In a first stage, an oxygenate feed is contacted with a first zeolite catalyst containing ZSM-5. The resulting conversion product contains an olefins composition. The olefin composition from the conversion reaction, with or without prior separation of ethylene and propylene, is then contacted with another zeolite catalyst in a second stage. The catalyst of such second stage is a one-dimensional zeolite having 10-membered ring channels, including ZSM-22, ZSM-23, ZSM-35, ZSM-48 or mixtures thereof. The eventual product comprises ethylene, propylene and C4+ olefins. The C4+ olefins may be partly recycled to the first stage as olefinic co-feed of the oxygenate feed. In the only example, pure methanol is converted by a two-step process into several olefins. In the process, typically use is made of a single reactor with a stacked bed configuration wherein the first stage is carried out in a first zeolite catalyst bed and the second stage is carried out a second zeolite bed (see FIG. 1 of U.S. Pat. No. 6,797,851). Alternatively, the two stages are carried out in two separate reactors (see FIGS. 2-5 of US B 797851).

The process of US B 797851 requires significant amounts of zeolite comprising catalysts. Consequently, the catalyst significantly contributes to the OPEX of the process.

It would be desirable to have a catalyst that is cost-efficient and contains high to maximum zeolite content.

### SUMMARY OF THE INVENTION

A molecular sieve catalyst is now proposed which is more attractive when compared with catalysts which are commonly used in processes to prepare olefins.

Accordingly, the present invention provides a molecular sieve catalyst which comprises a zeolitic material, a binder material and a matrix material, wherein the zeolitic material comprises zeolite and crystobolite, and the crystobolite is present in an amount of less than 1.0 wt %, based on the total weight of zeolite in the molecular sieve catalyst.

The molecular sieve catalyst in accordance with the present invention has various advantages over known catalysts. The present molecular sieve catalyst allows for the use of higher contents of zeolite which improves the overall activity of the catalyst, resulting in an improved productiveness of the olefins preparation process. The content of zeolitic material that can be admixed with the other catalyst components such as the binder and matrix material is limited. When too much of the zeolitic material is present compared to the binder and matrix material the structural integrity and strength of the resulting catalyst may be compromised or it may even be impossible to prepare a suitable catalyst particle. Typically, at most 45 wt %, or even preferably at most 40 wt % of zeolitic material may be added to the catalyst composition without compromising the catalyst. As it is beneficial to have as much zeolite as possible in the catalyst, it is preferable that zeolite content in the zeolitic material is as high as possible. In the present invention it is proposed to provide a zeolitic material with a high zeolite content by using a zeolitic material that has a low crystobolite content, thereby reducing the dilution of the zeolite content in the zeolitic material by any crystobolite present in the zeolitic material.

In addition, by controlling the amount of crystobolite, the zeolite content can be maximized in a highly efficient manner, allowing the process to be fine-tuned and to be carried out in a more cost-effective and efficient manner.

Hence, the molecular sieve catalyst according to the present invention establishes a very attractive overall process for the preparation of olefins such as ethylene and propylene.

### DETAILED DESCRIPTION OF THE INVENTION

In the present invention use is made of a molecular sieve catalyst in which the zeolitic material comprises crystobolite in an amount of less than 1.0 wt %, based on the total weight of zeolite in the molecular sieve catalyst.

Preferably, the crystobolite is present in an amount of less than 0.5 wt %, based on the total weight of zeolite in the molecular sieve catalyst. More preferably, the crystobolite is present in an amount in the range of from 0.0001-0.1 wt %, based on the total weight of zeolite in the molecular sieve catalyst.

Crystobolite may also be referred to by other spellings used by those of ordinary skill in the art. It may also be spelled as crystabolite or cristobalite.

The zeolitic material to be used in accordance with the present invention suitably comprises one or more zeolites. The molecular sieve catalysts also include a binder material, a matrix material and optionally fillers. Suitable matrix materials include clays, such as kaolin. Suitable binder materials include silica, alumina, silica-alumina, titania and zirconia, wherein silica is preferred due to its low acidity.

The zeolites preferably have a molecular framework of one, preferably two or more corner-sharing [TO₄] tetrahedral units, more preferably, two or more [SiO₄] and/or [AlO₄] tetrahedral units. These silicon and/or aluminum based molecular sieves and metal containing silicon and/or aluminum based molecular sieves have been described in detail in numerous publications including for example, U.S. Pat. No. 4,567,029. In a preferred embodiment, the molecular sieve catalysts have 8-, 10- or 12-ring structures and an average pore size in the range of from about 3 Å to 15 Å.

Preferably, the amount of zeolite in the molecular sieve catalyst is suitably from 20 to 50 wt %, preferably from 35 to 45 wt %, based on total weight of the molecular sieve catalyst composition.

Suitable zeolites include those of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

Preferred zeolites include more-dimensional zeolites, in particular of the MFI type, more in particular ZSM-5, or of the MEL type, such as ZSM-11. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably, the channels in at least one of the directions are 10-membered ring channels. A preferred MFI-type zeolite has a Silica-to-Alumina ratio (SAR) of at least 60, preferably at least 80.

One preferred catalyst may comprise one or more zeolites having one-dimensional 10-membered ring channels, i.e. one-dimensional 10-membered ring channels, which are not intersected by other channels.

Preferred examples are zeolites of the MTT and/or TON type.

In a particularly preferred embodiment the molecular sieve catalyst comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The present molecular sieve catalyst may comprise phosphorus as such or in a compound, i.e. phosphorous other than any phosphorus included in the framework of the molecular sieve. It is preferred that an MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the present molecular sieve catalyst comprising MEL or MFI-type zeolites comprises phosphorus as such or in a compound in an elemental amount of from 0.05-10 wt % based on the weight of the formulated catalyst. A particularly preferred catalyst comprises phosphorus and MEL or MFI-type zeolites having SAR of in the range of from 60 to 150, more preferably of from 80 to 100. An even more particularly preferred catalyst comprises phosphorus and ZSM-5 having SAR of in the range of from 60 to 150, more preferably of from 80 to 100.

It is preferred that the molecular sieves in the hydrogen form are, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve catalyst used is in the hydrogen form. It is well known in the art how to produce such molecular sieve catalysts in the hydrogen form.

The molecular sieve catalyst particles can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. Spherical particles are normally obtained by spray drying. Preferably the average particle size is in the range of 1-500 μm, preferably 50-100 μm.

Preferably, the zeolite comprises a zeolite having at least 10-membered ring channels. Preferred zeolites include zeolites of the MTT-type, the TON-type, the MFI-type or the MEL-type.

The present invention also provides a process for formulating a molecular sieve catalyst according to the present invention, which process comprises mixing the zeolitic material(s) with the binder material and the matrix material to form the molecular sieve catalyst.

The present invention further provides a process for the preparation of olefins, which process comprises reacting an oxygenate feed and/or olefinic feed in a reactor in the presence of the present molecular sieve catalyst to form an effluent comprising olefins.

Suitably, the process comprises the steps of:
(a) reacting the oxygenate feed and/or olefinic feed in a reactor in the presence of the molecular sieve catalyst according to the present invention to form the effluent comprising olefins;
(b) separating the effluent comprising olefins as obtained in step (a) into at least a first olefinic product fraction comprising ethylene and/or propylene and a second olefinic fraction comprising olefins having 4 or more carbon atoms; and optionally
(c) recycling at least part of the second olefinic fraction as an olefinic recycle stream to step (a). By recycling at least part of the second olefinic fraction as an olefinic recycle stream to step (a), the yield of ethylene and propylene may be increased.

In another embodiment, the process comprises the steps of:
(a) reacting the oxygenate and/or olefinic feed in a reactor in the presence of the molecular sieve catalyst according to the invention to form the effluent comprising olefins;
(b) fractionating the effluent comprising olefins as obtained in step (a) to obtain at least a first olefinic product fraction comprising ethylene and/or propylene, and a second olefinic product fraction comprising olefins having 4 or more carbon atoms;
(c) recycling at least part of the second olefinic product fraction as obtained in step (b) as recycle stream to step (a); and
(d) withdrawing at least part of the second olefinic product fraction as olefinic product.

In another attractive embodiment, the process comprises the steps of:
(a) reacting the oxygenate and/or olefinic feed in a reactor in the presence of the molecular sieve catalyst according to the invention to form the effluent comprising olefins;
(b) fractionating the effluent comprising olefins as obtained in step (a) to obtain at least a first olefinic product fraction comprising ethylene and/or propylene, and a second olefinic product fraction comprising olefins having 4 or more carbon atoms;
(c) fractionating the second olefinic product fraction as obtained in step (b) to obtain at least a third olefinic product fraction comprising C4 olefins and a fourth olefinic product fraction comprising olefins having 5 or more carbon atoms;
(d) recycling at least part of the third olefinic product fraction as obtained in step (c) as recycle stream to step (a);
(e) subjecting at least part of the fourth olefinic product stream to an olefin cracking process to form at least cracking effluent comprising ethylene and propylene; and
(f) recycling at least part of the cracking effluent comprising ethylene and propylene as formed in step (e) as recycle stream to step (b).

The present invention further provides a process which comprises the steps of:
(a) reacting the oxygenate and/or olefinic feed in a reactor in the presence of the molecular sieve catalyst according to the invention to form a mixture which comprises the effluent comprising olefins and at least partially coked catalyst;
(b) separating olefins and at least partially coked catalyst as obtained in step (a);
(c) recovering olefins obtained in step (b);
(d) passing at least partially coked catalyst as obtained in step (b) to a regenerator;
(e) introducing into the regenerator an oxygen-containing gas to regenerate at least part of the at least partially coked catalyst, thereby producing a gaseous mixture and at least partially regenerated catalyst;
(f) separating at least partially regenerated catalyst and at least part of the gaseous mixture as obtained in step (e); and
(g) passing at least part of the at least partially regenerated catalyst as obtained in step (f) to the reactor in step (a).

In the present invention, an oxygenate feed can be converted in an oxygenate-to-olefins (OTO) process or an olefinic feed can be converted in an olefin cracking process (OCP).

In step (a) an oxygenate feed and/or olefinic feed is introduced through introduction means into the reactor. The introduction means suitably comprises one or more devices that comprise one or more nozzles or open pipes or gas distributors.

In step (a), the oxygenate feed and/or olefinic feed is reacted in a reactor in the presence of the molecular sieve catalyst according to the invention to form a mixture which comprises olefins and at least partially coked catalyst. The reactor in step (a) can be an OTO reaction zone wherein the oxygenate feed is contacted with the molecular sieve catalyst under oxygenate conversion conditions, to obtain a conversion effluent comprising lower olefins. Reference herein to an oxygenate feed is to an oxygenate-comprising feed. In the OTO reaction zone, at least part of the feed is converted into a product containing one or more olefins, preferably including lower olefins, in particular ethylene and typically propylene.

The oxygenate used in the process according to the invention is preferably an oxygenate which comprises at least one oxygen-bonded alkyl group. The alkyl group preferably is a C1-C5 alkyl group, more preferably C1-C4 alkyl group, i.e. comprises 1 to 5, respectively, 4 carbon atoms; more preferably the alkyl group comprises 1 or 2 carbon atoms and most preferably one carbon atom. Examples of oxygenates that can be used in the oxygenate feed include alcohols and ethers. Examples of preferred oxygenates include alcohols, such as methanol, ethanol, propanol; and dialkyl ethers, such as dimethylether, diethylether, methylethylether. Preferably, the oxygenate is methanol or dimethylether, or a mixture thereof. More preferably, the oxygenate feed comprises methanol or dimethylether.

Preferably the oxygenate feed comprises at least 50 wt % of oxygenate, in particular methanol and/or dimethylether, based on total hydrocarbons, more preferably at least 70 wt %.

The oxygenate feed can comprise an amount of diluent, such as nitrogen and water, preferably in the form of steam. In one embodiment, the molar ratio of oxygenate to diluent is between 10:1 and 1:10, preferably between 4:1 and 1:2, in particular when the oxygenate is methanol and the diluent is water (steam).

A variety of OTO processes is known for converting oxygenates such as for instance methanol or dimethylether to an olefin-containing product, as already referred to above. One such process is described in WO-A 2006/020083. Processes integrating the production of oxygenates from synthesis gas and their conversion to light olefins are described in US20070203380A1 and US20070155999A1.

The reaction conditions of the oxygenate conversion in step (a) include a reaction temperature of 350 to 1000° C., suitably from 350 to 750° C., preferably from 450 to 750° C., more preferably from 450 to 700° C., even more preferably 500 to 650° C.; and a pressure suitably from 1 bara to 50 bara, preferably from 1-15 bara, more preferably from 1-4 bara, even more preferably from 1.1-3 bara, and most preferably in from 1.3-2 bara.

Suitably, the oxygenate-comprising feed is preheated to a temperature in the range of from 120 to 550° C., preferably 250 to 500° C. prior to introducing it into the reactor in step (a).

Preferably, in addition to the oxygenate, an olefinic co-feed is provided along with and/or as part of the oxygenate feed. Reference herein to an olefinic co-feed is to an olefin-comprising co-feed. The olefinic co-feed preferably comprises C4 and higher olefins, more preferably C4 and C5 olefins. Preferably, the olefinic co-feed comprises at least 25 wt %, more preferably at least 50 wt %, of C4 olefins, and at least a total of 70 wt % of C4 hydrocarbon species. The olefinic co-feed can also comprise propylene.

The reaction in step (a) may suitably be operated in a fluidized bed, e.g. a dense, turbulent or fast fluidized bed or a riser reactor or downward reactor system, and also in a fixed bed reactor, moving bed or a tubular reactor. A fluidized bed, e.g. a turbulent fluidized bed, fast fluidized bed or a riser reactor system are preferred.

The superficial velocity of the gas components in a dense fluidized bed will generally be from 0 to 1 m/s; the superficial velocity of the gas components in a turbulent fluidized bed will generally be from 1 to 3 m/s; the superficial velocity of the gas components in a fast fluidized bed will generally be from 3 to 5m/s; and the superficial velocity of the gas components in a riser reactor will generally be from 5 to about 25 m/s.

It will be understood that dense, turbulent and fast fluidized beds will include a dense lower reaction zone with densities generally above 300 kg/m³. Moreover, when working with a fluidized bed several possible configurations can be used: (a) co-current flow meaning that the gas (going upward) and the catalyst travels through the bed in the same direction, and (b) countercurrent, meaning that the catalyst is fed at the top of the bed and travels through the bed in opposite direction with respect to the gas, whereby the catalyst leaves the vessel at the bottom. In a conventional riser reactor system the catalyst and the vapours will travel co-currently.

More preferably, a fluidized bed, in particular a turbulent fluidized bed system is used. Suitably, in such a moving bed reactor the oxygenate feed is contacted with the molecular sieve catalyst at a weight hourly space velocity of at least 1 hr⁻¹, suitably from 1 to 1000 hr⁻¹, preferably from 1 to 500 hr⁻¹, more preferably 1 to 250 hr⁻¹, even more preferably from 1 to 100 hr⁻¹, and most preferably from 1 to 50 hr⁻¹.

The reactor in step (a) can also be an OCP reaction zone wherein the olefinic feed is contacted with a zeolite-comprising catalyst under olefin conversion conditions. Suitably, the olefinic feed comprises C4+ olefins are converted by contacting such a feed with a zeolite-comprising catalyst, thereby converting at least part of the olefins comprising 4 or more carbon atoms to olefins having a lower carbon number, i.e. a olefin having n carbon atoms is cracked to at least one olefin have m carbon atoms, wherein n and m are integers and m is smaller than n. Preferably, the olefins comprising 4 or more carbon atoms are cracked to at least ethylene and propylene.

Preferably, the olefinic feed is contacted with the zeolite-comprising catalyst in step (a) at a reaction temperature of 350 to 1000° C., preferably from 375 to 750° C., more preferably 450 to 700° C., even more Preferably 500 to 650° C.; and a pressure from 1 bara to 50 bara, preferably from 1-15 bara. Optionally, such olefinic feed also contains a diluent. Examples of suitable diluents include, but are not limited to, such as water or steam, nitrogen, paraffins and methane. Under these conditions, at least part of the olefins in the olefinic feed are converted to further ethylene and/or propylene.

The preferences provided herein above for the oxygenate to olefins catalyst apply mutatis mutandis for the OCP catalyst with the primary exception that the OCP catalyst always comprises at least one zeolite. Particular preferred catalysts for the OCP reaction, i.e.

converting part of the olefinic product, and preferably part of the C4+ hydrocarbon fraction of the olefinic product including olefins, are catalysts comprising at least one zeolite selected from MFI, MEL, TON and MTT type zeolites, more preferably at least one of ZSM-5, ZSM-11, ZSM-22 and ZSM-23 zeolites.

The catalyst may further comprise phosphorus as such or in a compound, i.e. phosphorus other than any phosphorus included in the framework of the molecular sieve. It is preferred that a MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05 to 10 wt % based on the weight of the formulated catalyst. A particularly preferred catalyst comprises phosphorus and MEL or MFI-type zeolite having SAR of in the range of from 60 to 150, more preferably of from 80 to 100. An even more particularly preferred catalyst comprises phosphorus and ZSM-5 having SAR of in the range of from 60 to 150, more preferably of from 80 to 100.

Preferably, the oxygenate to olefins catalyst and the olefin cracking catalyst are the same zeolite-comprising catalyst.

Also an OCP process may suitably be operated in a fluidized bed, e.g. a fast fluidized bed or a riser reactor or a downward reactor system, and also in a fixed bed reactor, moving bed or a tubular reactor. A fluidized bed, e.g. a fast fluidized bed or a riser reactor system are preferred.

In step (c), at least part of the second olefinic fraction which comprises olefins having 4 or more carbon atoms is recycled to step (a) for use as an olefinic co-feed.

Preferably, at least 70 wt % of the olefinic co-feed, during normal operation, is formed by the recycle stream of the second olefinic fraction containing olefins having 4 or more carbon atoms, preferably at least 90 wt % of olefinic co-feed, based on the whole olefinic co-feed, is formed by such a recycle stream. In order to maximize production of ethylene and propylene, it is desirable to optimize the recycle of C4 olefins in the effluent of the OTO or olefinic cracking process. This can be done by recycling at least part of second olefinic fraction containing olefins having 4 or more carbon atoms, preferably the C4-C5 hydrocarbon fraction, more preferably the C4 hydrocarbon fraction, to the OTO reaction zone in step (a). Suitably, however, a certain part thereof, such as between 1 and 5 wt %, is withdrawn as purge, since otherwise saturated hydrocarbons, in particular C4's (butane) would build up in the process, which are substantially not converted under the OTO or OCP reaction conditions.

The preferred molar ratio of oxygenate in the oxygenate feed to olefin in the olefinic co-feed provided to the OTO reaction zone in step (a) depends on the specific oxygenate used and the number of reactive oxygen-bonded alkyl groups therein. Preferably the molar ratio of oxygenate to olefin in the total feed lies in the range of 20:1 to 1:10, more preferably in the range of 18:1 to 1:5, still more preferably in the range of 15:1 to 1:3, even still more preferably in the range of 12:1 to 1:3.

Although the second olefinic fraction containing olefins having 4 or more carbon is recycled as an olefinic co-feed to the OTO reaction zone in step (a), alternatively at least part of the olefins in this second olefinic fraction is converted to ethylene and/or propylene by contacting such C4+ hydrocarbon fraction in a separate OCP unit with a zeolite-comprising catalyst.

Preferably, the C4+ hydrocarbon fraction is contacted with the zeolite-comprising catalyst at a reaction temperature of 350 to 1000° C., preferably from 375 to 750° C., more preferably 450 to 700° C., even more Preferably 500 to 650° C.; and a pressure from 1 bara to 50 bara, preferably from 1-15 bara. Optionally, such a stream comprising C4+ olefins also contains a diluent. Examples of suitable diluents include, but are not limited to, such as water or steam, nitrogen, paraffins and methane. Under these conditions, at least part of the olefins in the C4+ hydrocarbon fraction are converted to further ethylene and/or propylene. The further ethylene and/or propylene may be combined with the further ethylene and/or propylene as obtained in step (b). Such a separate process step directed at converting C4+ olefins to ethylene and propylene is, as will be clear from the foregoing, also referred to as an olefin cracking process (OCP).

In such a subsequent separate OCP suitably zeolite-comprising catalysts are used. Catalysts suitable for an olefin cracking process have been described herein above and may be used for the additional separate OCP process step.

Preferably, the catalyst used in step (a) and the separate OCP unit are the same zeolite-comprising catalyst.

Preferably, at least part of the second olefinic product fraction comprising olefins having 4 or more carbon atoms as obtained in step (b) is first fractionated to obtain at least a third olefinic product fraction comprising C4 olefins and a fourth olefinic product fraction comprising olefins having 5 or more carbon atoms, wherein at least part of the fourth fraction is provided to the separate olefin cracking unit, while at least part of the third olefinic product fraction is recycled as recycle stream to step (a). By converting the olefins having 5 or more carbon atoms separately in the separate OCP unit, the conditions in the separate OCP unit can be selected to obtain an optimal converse of olefins having more than 5 carbon atoms.

Also the subsequent OCP process may suitably be operated in a fluidized bed, e.g. a fast fluidized bed or a riser reactor or downward reactor system, and also in a fixed bed reactor, moving bed or a tubular reactor. A fluidized bed, e.g. a fast fluidized bed or a riser reactor system are preferred.

The olefinic product obtained from the OTO process in step (a) comprises ethylene and/or propylene, which may be separated from the remainder of the components in the olefinic product. When the olefinic product comprises ethylene, at least part of the ethylene may be further converted into at least one of polyethylene, mono-ethylene-glycol, ethylbenzene and styrene monomer. When the olefinic product comprises propylene, at least part of the propylene may be further converted into at least one of polypropylene and propylene oxide.

Preferably, the olefins as recovered in step (b) are subjected to a quenching treatment before they are separated into at least first and second olefinic fraction. In such a quenching treatment water and C6+ hydrocarbons can be removed from the olefins are subjected to a fractionating treatment to separate the olefins into at least the first olefinic product fraction containing ethylene and/or propylene and the second olefinic fraction containing olefins having 4 or more carbon atoms.

Suitably, the olefins are subjected to a heat recovery step before they are subjected to the quenching treatment.

More preferably, olefins obtained after the quenching treatment are first compressed before they are separated into at least the first olefinic fraction and the second olefinic fraction.

Instead of a quenching treatment of the olefins, also use can be made of air coolers to bring down the temperature of the olefins.

In one of the above described embodiments comprising a regeneration step in step (d) at least partially coked catalyst as obtained in step (b) is passed to a regenerator. Suitably, the at least partially coked catalyst as obtained in step (b) is passed in its entirety or a portion of it to the regenerator. The molecular sieve catalyst to be used in accordance with the present invention deactivates in the course of the process with time, due to issues around coke deposition and hydrothermal stability. Hence, the molecular sieve catalyst needs to be regenerated in order to at least partly remove coke from the coked catalyst as obtained in step (b). Conventional catalyst regeneration techniques can be employed to remove the coke. It is not necessary to remove all the coke from the catalyst as it is believed that a preset amount of residual coke may enhance the catalyst performance and additionally, it is believed that complete removal of the coke may also lead to degradation of the molecular sieve.

In order to regenerate at least part of the at least partially coked catalyst an oxygen-containing gas is introduced in the regenerator in step (e), thereby producing a gaseous mixture and at least partially regenerated catalyst. The oxygen-containing gas may be chosen from oxygen and air. Also mixtures can suitably be used of these oxygen-containing gases. Preferably, the oxygen-containing gas comprises oxygen, more preferably air is used as the oxygen-containing gas.

In step (e), the regeneration is carried out under conditions of temperature, pressure and residence time that is usually applied in regeneration processes to burn coke from catalysts. Suitably, between 0.01-5 wt % of the coke present on the at least partially coked catalyst is removed from the catalyst during regeneration.

Suitably, the regeneration in step (e) is carried out at a temperature in the range of from 580-800° C., preferably in the range of from 600-750° C., more preferably in the range of from 620-680° C., and a pressure in the range of from 1-5 bara, preferably in the range of from 1-3 bara, more preferably in the range of from 1.3-2 bara. The regeneration can suitably be carried out in a fixed bed, or a fluidized bed such as a dense, turbulent or fast fluidized bed, or in a riser regenerator. Preferably, the regeneration is carried out in a turbulent fluidized bed.

Suitably, the regeneration in step (e) can be carried out in a periodical manner or continuous manner. Preferably, the regeneration in step (e) is carried out in a continuous manner.

In a further aspect the invention provides a molecular sieve catalyst which comprises a zeolite, a binder material and a matrix material, wherein the catalyst comprises crystobolite in an amount of less than 1.0 wt %, based on the total weight of zeolite in the molecular sieve catalyst. Preferably, the catalyst comprises crystobolite in an amount in the range of from 0.0001-0.1 wt %, based on the total weight of zeolite in the molecular sieve catalyst.

1. The molecular sieve catalyst which comprises a zeolitic material, a binder material and a matrix material, wherein the zeolitic material comprises zeolite and crystobolite, and the crystobolite is present in an amount of less than 1.0 wt %, based on the total weight of zeolite in the molecular sieve catalyst.

2. The catalyst according to claim 1, wherein the crystobolite is present in an amount of less than 0.5 wt %, based on the total weight of zeolite in the molecular sieve catalyst.

3. The catalyst according to claim 1, wherein the crystobolite is present in an amount in the range of from 0.0001-0.1 wt %, based on the total weight of zeolite in the molecular sieve catalyst.

4. The catalyst according to claim, wherein the zeolitic material comprises a zeolite having at least 10-membered ring channels.

5. The catalyst according to claim 4, wherein the zeolite is a zeolite of the MTT-type, the TON-type, the MFI-type or the MEL-type.

6. The process for the preparation of olefins, which process comprises reacting an oxygenate and/or olefinic feed in a reactor in the presence of a molecular sieve catalyst as defined in claim 1 to form an effluent comprising olefins.

7. The process according to claim 6, wherein the process comprises the steps of:
(a) reacting the oxygenate feed and/or olefinic feed in a reactor in the presence of the molecular sieve catalyst to form the effluent comprising olefins;
(b) separating the effluent comprising olefins as obtained in step (a) into at least a first olefinic product fraction comprising ethylene and/or propylene and a second olefinic fraction comprising olefins having 4 or more carbon atoms; and
(c) recycling at least part of the second olefinic fraction as an olefinic recycle stream to step (a).

8. The process according to claim 6, wherein the oxygenate feed comprises methanol and/or dimethylether.

9. The process according to claim 6, wherein the reaction in step (a) is conducted at a temperature from 350 to 750° C.

10. The molecular sieve catalyst which comprises a zeolite, a binder material and a matrix material, wherein the catalyst comprises crystobolite in an amount of less than 1.0 wt %, based on the total weight of zeolite in the molecular sieve catalyst.

11. The catalyst according to claim 10, wherein the catalyst comprises crystobolite in an amount in the range of from 0.0001-0.1 wt %, based on the total weight of zeolite in the molecular sieve catalyst.
